(19) United States
(12) Patent Application Publication
(10) Pub. No.: US 2021/0177349 A1
(43) Pub. Date: Jun. 17, 2021

(54) A Wearable Diagnostic Device for Measuring Third Party Vitals

(71) Applicants: Suraj Mohan, Chennai (IN); Anupama Vasudevan, Chennai (IN)

(72) Inventors: Suraj Mohan, Chennai (IN); Anupama Vasudevan, Chennai (IN)

(21) Appl. No.: 16/964,543

(22) PCT Filed: Aug. 8, 2018
(86) PCT No.: PCT/IN2018/050517

§ 371 Date: Dec. 22, 2020

(30) Foreign Application Priority Data
Jan. 24, 2018 (IN) 201841002884

(51) Int. Cl. A61B 5/00; A61B 5/024; A61B 5/0255; A61B 5/145

(52) U.S. Cl. CPC A61B 5/6806; A61B 5/0008; A61B 5/0011; A61B 5/02411; A61B 2562/046; A61B 5/14532; A61B 5/14542; A61B 5/742; A61B 5/0255

(57) ABSTRACT

A method and a system for measuring various physical parameters of a subject by a user using a wearable diagnostic device which comprises of an apparel with a modular sensor matrix disposed in it. The modular sensor matrix is configured to enable a user to measure physical parameters of the subject. The apparel also has display visual display unit disposed on it, which is configured to exhibit measured physical parameters of the subject.

### TECHNICAL FIELD

The invention generally relates to wearable diagnostic device and more specifically the invention provides a system and method of measuring various physical parameters of a third party using a diagnostic device.

### BACKGROUND ART

Wearable technology, also referred to as wearable gadgets, can be defined as electronics that can be worn on the human body. These can either be accessories or apparel. Examples include ‘smart’ bands, watches, glasses, ‘smart’ clothing etc. The main features of wearable technology include the ability to automatically measure parameters, record information, process data, connect to the internet and data exchange (mostly wireless) with other devices. These accessories or apparel are referred to as ‘smart’ accessories or apparel due to their capability to perform the aforementioned functions.

Wearable technologies have taken the ‘smart’ accessory industry by storm by its exponential growth mostly in the field of health and fitness. These devices help a user to track workout, calorie count, heart rate, sleep pattern, step count and other parameters of the user who wants to keep a track of such activity. A user may even sync wearable tech accessories to a mobile phone to perform a range of other functions including viewing notifications, controlling music, and navigating through maps.

Attempts have been made to utilize these smart accessories in the field of diagnostics. Given that these smart accessories measure physical parameters automatically and on a continuous basis, stakeholders in the health care space are keen to utilize these accessories beyond just fitness activity tracking. Newer technologies integrating diagnostics with wearable technology have started surfacing. These technologies include several features where sensors are mounted on wearables (wrist band, t-shirts, sleeves etc.) to measure several parameters like human blood pressure, pulse rate, temperature, respiration rate, blood oxygen, skin resistance, motion analysis etc. Once these parameters are measured, the results are collected, processed and transmitted to other devices.

However, it can't be ignored that existing methods and wearable gadgets have failed to effectively utilize the facilities provided by advances in wearable technology. One major drawback that can be pointed out is that most of the wearable gadgets have no provision of one user (doctor or medical professional) examining another person as most of these gadgets involve methods of user wearing the gadget to measure his/her own health parameters.

Another drawback of the existing wearable gadgets is that these gadgets do not simultaneously measure physical parameters and the users need to toggle through various options within the gadgets to measure any specific parameter of choice.

Hence there is a need for an effective diagnostic device that can enable a user to measure multiple health parameters of another person where such parameters are measured simultaneously. Effective measuring of several parameters like foetal heart rate, venous pressure measurement, blood sugar levels, haemoglobin, body temperature, saturation and many more need to be covered under one device.

### OBJECT OF INVENTION

The object of the invention is to measure various physical parameters of a person by another user, using a wearable diagnostic device.

Another object of the invention is to provide a modular sensor matrix which is configured to simultaneously measure multiple physical parameters of a user.

Yet another object of the invention is to provide a wearable diagnostic device which is customizable in terms of input frequency, wavelength, speed and the like.

Yet another object of the object of the invention is to provide a wearable diagnostic device which is configured to support modularity.

Yet another object of the invention is to provide an optical wireless communication method.

### SUMMARY OF INVENTION

The invention provides a wearable diagnostic device for measuring various physical parameters of a subject by a user, which comprises of an apparel with a modular sensor matrix disposed on it. The modular sensor matrix is configured to enable a user to measure physical parameters of said subject. The apparel further comprises a display visual display unit disposed on it, which is configured to exhibit measured physical parameters of the subject. Further, the system may have a wireless communication component configured to permit the wearable diagnostic device to wirelessly communicate and transfer data to other devices.

These and other aspects of the embodiments herein will be better appreciated and understood when considered in conjunction with the following description and the accompanying drawings.

### BRIEF DESCRIPTION OF DRAWINGS

This invention is illustrated in the accompanying drawings, throughout which, like reference letters indicate corresponding parts in the various figures.

The embodiments herein will be better understood from the following description with reference to the drawings, in which:

FIG. 1 shows the system depicting working of the diagnostic device (glove), in accordance with the current invention.

FIG. 2 shows front side (palm side) design of a wearable diagnostic device (glove) for simultaneous measuring of physical parameters, in accordance with an embodiment of the present invention.

FIG. 3 shows back side design of a wearable diagnostic device (glove) with a display of physical parameter data, in accordance with an embodiment of the present invention.

FIG. 4 shows details of a holder integrated with the wearable diagnostic device (glove) and placement of sensor matrices into it respectively, in accordance with an embodiment of the present invention.

FIG. 5 shows data acquisition system of the wearable diagnostic device, in accordance with an embodiment of the present invention.

FIG. 6 shows the visual display unit associated with the diagnostic device system, in accordance with an embodiment of the present invention.

FIG. 7 provides a flowchart illustrating the method of measuring physical parameters of a subject by a user, in accordance with an embodiment of the present invention.

### DESCRIPTION OF EMBODIMENTS

The embodiments herein and the various features and advantageous details thereof are explained more fully with reference to the non-limiting embodiments that are illustrated in the accompanying drawings and/or detailed in the following description. Descriptions of well-known components and processing techniques are omitted so as to not unnecessarily obscure the embodiments herein. The examples used herein are intended merely to facilitate an understanding of ways in which the embodiments herein may be practised and to further enable those of skill in the art to practice the embodiments herein. Accordingly, the examples should not be construed as limiting the scope of the embodiments herein.

The main objective of the invention is to measure various physical parameters of a person with the help of a wearable diagnostic device worn by a user other than the subject whose physical parameters are being measured. The diagnostic device is configured with a modular sensor matrix and a visual display unit. The device is highly customizable in terms of input frequency, wavelength, speed etc. allowing host physical parameters to be measured for different types of diagnosis. The diagnostic device is configured to support modularity and built with the ability to communicate with other devices.

In the present disclosure, modular sensor matrix may be referred to as a systematic arrangement of sensors within the diagnostic device.

In the present disclosure, physical parameters that may be measured with the wearable diagnostic device may be haemoglobin, blood sugar level, oxygen saturation, pulse-rate, oxymetry, foetal rate, wheezing, cardiac murmurs, anaemia and the like.

While the term wearable diagnostic device is intended to cover any wearable apparel capable of being worn on the human body, for the purposes of illustration a glove as a wearable diagnostic device has been discussed throughout this document.

Referring now to the drawings, where similar reference characters denote corresponding features consistently throughout the figures, there are shown preferred embodiments.

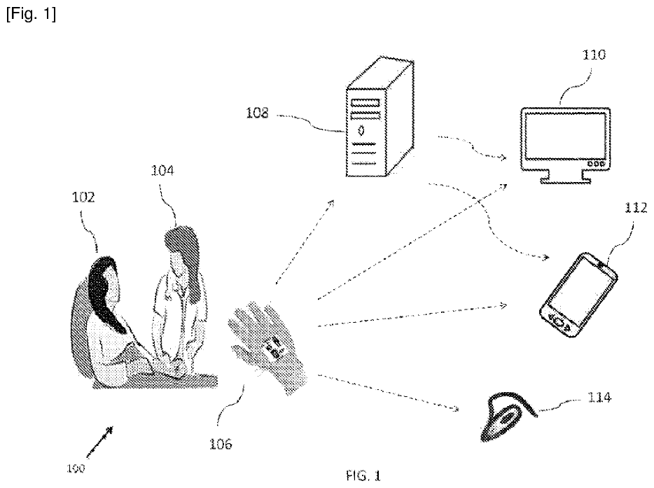

FIG. 1 displays the system 100 that illustrates a surgical glove 106 as a wearable diagnostic device. A user 104 wears the surgical glove 106 in order to measure the physical parameters of a subject 102. The surgical glove 106 is brought in close proximity of the subject 102. The glove may include a modular sensor matrix containing multiple sensors to measure various physical parameters of the subject 102 simultaneously. Various measured parameters can be exhibited on the display visual display unit provided on the glove 106. The diagnostic device may contain a rechargeable battery (not shown in the figure) to power up the modules disposed within/integrated to it.

Encouraged by the advances in wireless communication, the diagnostic device may also include optical wireless communication module (not shown in figure) enabling the sensors to communicate via optical wireless communication method with the visual display unit provided on the glove 106 and to connect to server/cloud storage and hence measured data can be wirelessly communicated to various devices like a server 108, a computer system 110, a mobile device 112 or even a Bluetooth device 114. Moreover, the optical wireless communication module enables a facility such as a hospital to communicate with the diagnostic device and patient's smart wearable (such as watch, mobile, band etc) providing navigation to various examination rooms (such as for X-ray, blood test etc) within the hospital, and transferring the measured data at the end of each test back to the diagnostic device (or a central data base) for the doctor's perusal.

Further, the data stored in the server 108 can be retransmitted to any other devices. The data can even be sent directly to the subject's 102 smart band or mobile device 112 enabling easier transmission and storage of data while directly reducing the wastage of paper based health records and prescriptions.

In an alternative embodiment, surgical glove can be replaced by other apparel like a wrist band or it could be a sleeve within which the modular sensor matrix and the visual display unit may be disposed.

In one embodiment, the optical wireless communication module may include LiFi technology, the pings from various light fixtures within a premise.

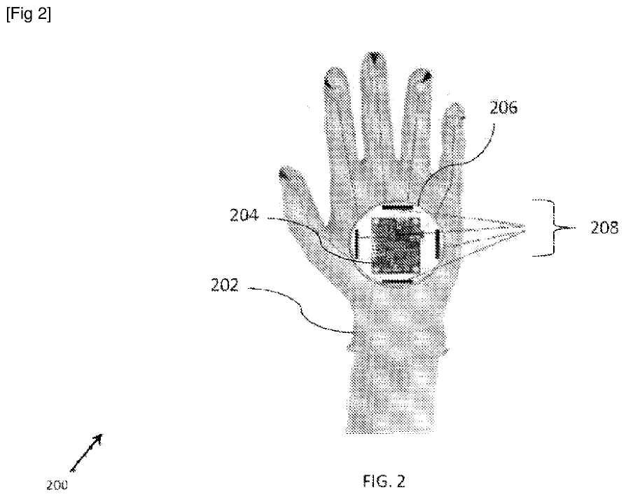

FIG. 2 shows the front design (palm side) 200 of a wearable diagnostic device (glove) 202, in accordance with an embodiment of the present invention. The sensors constituting the modular sensor matrix 204 include any sensors used for measuring physical parameters whether such sensors are known now or developed in the future. These sensors are designed to simultaneously measure physical parameter of the subject. The glove 202 has a holder 206 integrated in the middle of it to accommodate the modular sensor matrix 204 with the help of supports 208 that are made of magnet and the like. Each sensor is designed to measure different physical parameters of the subject, for instance, a specified sensor from the modular sensor matrix 204 is configured to measure oxygen saturation in the body of a subject, another sensor is designed to measure blood sugar level, yet another sensor measures pulse-rate and another sensor measures haemoglobin data and wheezing and the like.

Further, sensor matrix 204 may have a sensor that can be used to measure foetal heart rate of a prenatal baby inside a mother's womb. Development of a baby at every stage from gestational age to birth can be tested and heart rate at every stage can be measured using this sensor. Since the modular sensor matrix 204 has the capability of measuring the physical parameters of a subject wherein multiple parameters are measured simultaneously, a doctor can measure the physical parameters of any patient efficiently simply by bringing the glove 202 in close proximity of the patient.

Furthermore, the glove 202 is configured with the provision or an input channel where the input variables required for measuring physical parameters of a subject may be varied/adjusted as per need based on what particular types of physical parameters are required to be measured. This provision is highly demanded because specific physical parameters are displayed only at specific range/value of those input variables. For instance, using the sensor pulse oxymeter, a user may measure parameters such as haemoglobin, oxygen saturation, sugar level by customizing the wavelength. In one embodiment, the sensor matrix 202 may have the input channel (not shown in figure) such as knob, button and the like for changing/adjusting of input variables.

In one embodiment, input variables may be frequency, wavelength, speed and the like.

In one embodiment, the glove 202 may be configured with an integrated camera (not shown in figure) that acts as a high resolution image capture mechanism or as an X-ray scanner and fetch the x-ray images onto the display. The camera may be capable of measuring minute parameters for example those associated with the human retina.

In one embodiment of present invention, data transmission and navigation may happen through other wireless communication module such as WiFi, Bluetooth, NFC, GPS etc. in absence of optical wireless communication module.

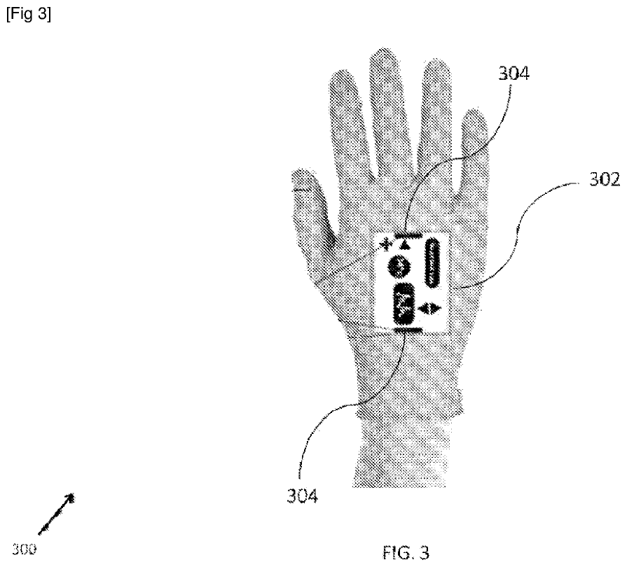

FIG. 3 shows back side design 300 of a wearable diagnostic device (glove) 202 with a display of physical parameter data, in accordance with an embodiment of the present invention. data collected from the modular sensor matrix 204 (shown in FIG. 2) of the diagnostic device is processed and transmitted wirelessly through optical wireless module to the visual display unit 302 which is shown in the FIG. 3. The visual display unit 302 may be OLED based or may be any general visual display unit used in hospitals and medical institutions to measure and monitor physical parameters. There are supports 304 made of magnet or similar substance in the edges of the visual display unit 302 to keep the visual display attached to the diagnostic device. The visual display unit 302 shows all the physical parameters transmitted from the modular sensor matrix 204, e.g., display oxygen saturation of the subject 102 or display of the blood sugar level, pulse rate, foetal heart rate or even the x-ray scanned image (not shown in the fig). In an embodiment, the modular sensor matrix is wirelessly connected to the visual display unit 302 thereby ensuring ease of usage.

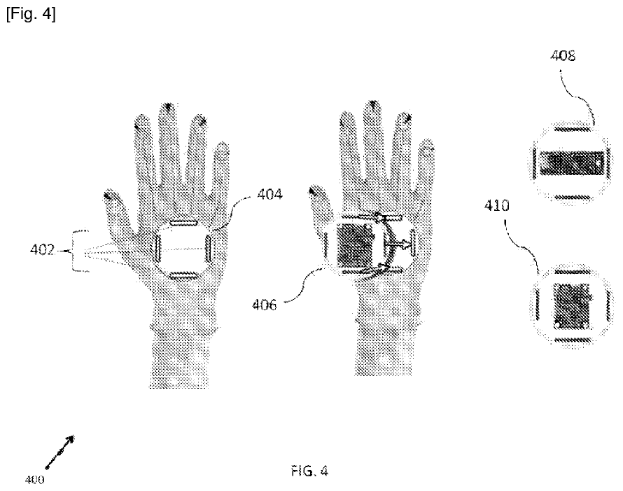

FIG. 4 shows front view 400 of the holder 404 integrated with the wearable diagnostic device (glove) and placement of modular sensor matrix 406 into the holder 404, in accordance with an embodiment of the present invention. The supports 402, made of magnet or similar material, aids in inserting and fitting of the modular sensor matrix 406 into the holder 404. The modular sensor matrix 406 may be ergonomic designs which may enable them to be detachably integrated into disposable surgical gloves (or any other apparel) with suitable provisions to insert the devices and remove them in such a manner that single diagnostic device maybe used across multiple patients while maintaining expected hygiene levels.

Different types of sensor matrices 408 or 410 which may be integrated to the diagnostic device (glove) for the purpose of using in measuring various physical parameters of a subject are shown in FIG. 4. Such a sensor matrix may be disposed with a data processing unit and communication module along with different sensors where each sensor may measure different physical parameters based on the input provided by the user via touch visual display unit display technologies such as OLEDs, or an individual sensor may indicate different/multiple health parameters.

In a preferred embodiment, data processing unit may be disposed in the server 108. In this embodiment, measured data from the modular sensor matrix 204 are transmitted to the server 108 via optical wireless communication module and then after data processing unit accomplishes its functions in the server, the processed data are sent back to the display unit of the diagnostic device.

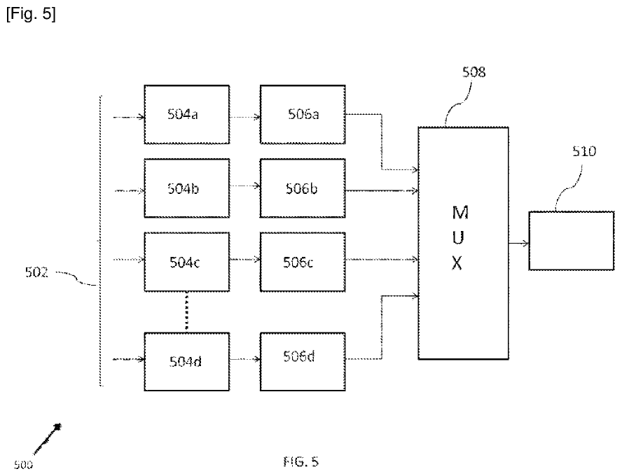

FIG. 5 represents data acquisition system of the wearable diagnostic device. The diagnostic device is taken into close proximity of the subject 202. Different physical parameters 502 of the subject 202 are fed to different transducers 504n where transducer series 504a to 504d converts physical parameters to electrical signals. These signals are further fed to a series of signal conditioners 506n (506a to 506d), where said electrical signals are converted to required form of electrical signals. Basically signal conditioners convert an electrical signal that may be difficult to read by conventional instrumentation into a more easily readable format. Such upgraded electrical signals are sent to multiplexer (mux) 508. Mux selects one signal out of various analog signals and forwards it into a single line, which leads to display 510. And thus selected analog data is displayed on the visual display 510.

In an alternative embodiment, said analog signals may be sent to A/D converter which converts analog signals to digital signals. Once converted, digital signals can be transmitted and viewed in various forms. Digital signals can be printed or displayed digitally or can be recorded using any recording media now known or developed in the future.

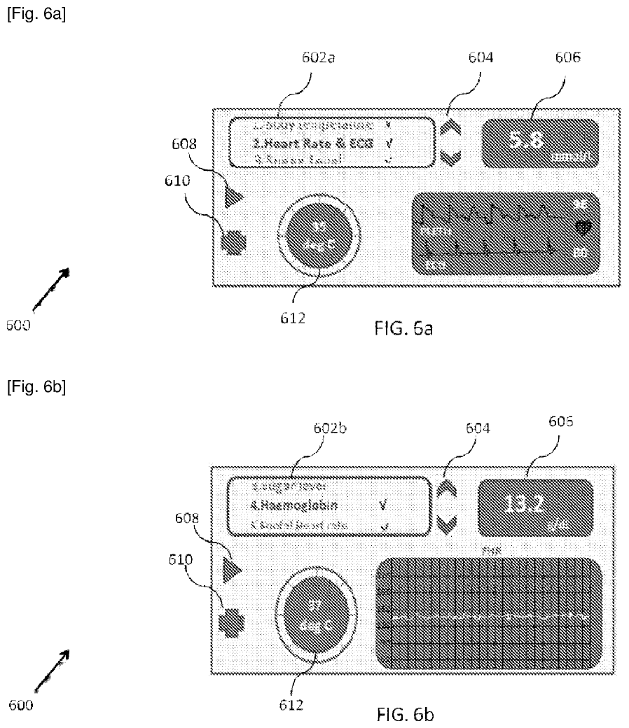

In one embodiment 600, FIGS. 6a and 6b shows the structure and design of the display associated with the diagnostic device system. The display mainly consists of a window which exhibits different physical parameters (602a & 602b) lined up at the top left corner, among which required physical parameter can be selected to measure, an action button 604 is provided to navigate between the different physical parameters. The navigation action is also shown in the embodiment, where FIG. 6a shows that heart rate and ECG parameter is selected 602a and the measurement is shown at the top right corner window 606. In the next instance, Haemoglobin parameter 602b is selected using the action button 604, which is shown in the FIG. 6b. The display further consists of a return/back button 608 to go previous step and a home button 610 come out of all the steps. The display furthermore consists of a window which exhibits body temperature 612.

Detailed Description on Measuring Procedure of Different Physical Parameters:

Electric foetal heart rate monitoring: A transducer is moved over the area being tested and high-frequency sound waves are transmitted from the probe through the gel into the body. The transducer collects the sounds that bounce back and a computer then uses those sound waves to create an image (2-MHz or 3-MHz probes). Most practitioners can find the heart rate with either probe. A 3-MHz probe is recommended to detect a heart rate in early pregnancy (8-10 weeks gestation). A 2-MHz probe is recommended for pregnant women who are overweight. Newer 5-MHz transvaginal probes aids in the detection of foetal heart tones (FHT) early in pregnancy (6-8 weeks) and for patients who have a retroverted uterus or throughout pregnancy for FHT detection for women who are obese.

Pulse oximeter: Pulse oximeters consist of two light emitting diodes, at 600 nm and 940 nm, and two light collecting sensors, which measure the amount of red and infra-red light emerging from tissues traversed by the light rays. The relative absorption of light by oxyhemoglobin (HbO) and deoxyhemoglobin is processed by the device and an oxygen saturation level is reported. The device directs its attention at pulsatile arterial blood and ignores local noise from the tissues. The result is a continuous qualitative measurement of the patients' oxyhemoglobin status. Oxygenated blood absorbs light at 660 nm (red light), whereas deoxygenated blood absorbs light preferentially at 940 nm (infra-red).

Sugar levels: From the pulse oxymeter, we can get the sugar levels, if sugar level is high the density of blood is more, if sugar levels are low, the density of blood is less, if we use 2 LEDs at 600 nm and 940 nm, the LED with wavelength 600 nm will isolate oxygenated blood. From this we can get the wavelength of red light emerging at the other end. The wavelength will be more if the blood sugar is low and wavelength will be very less if the blood sugar is high. The range though has to be determined for an individual.

Temperature sensors: Temperature sensors are often built from electronic components called thermistors. A thermistor is a device whose resistance varies with temperature (the name comes from a combination of the terms “thermal” and “resistor”). Typical thermistors are made from ceramic semiconductors or from platinum wires wrapped around ceramic mandrels or spindles. Thermistors usually have negative temperature coefficients (NTC), meaning the resistance of the thermistor decreases as the temperature increases. Depending on the material and fabrication process, the typical operating range for thermistors is −50° C. to 150° C. The small size of most thermistors results in a rapid response to temperature changes. A thermistor requires a calculation involving a natural log, which can consume a lot of computational cycles and code space in the micro-controller.

Haemoglobin and Anaemia: Uses a non-invasive optical measurement platform combined with a finger attached ring-shaped sensor probe. The pressure applied by the sensor temporarily occludes the blood flow in the finger, creating new blood dynamics which generate a unique, strong optical signal, yielding a high signal-to-noise ratio which is wholly blood specific. Analysis of the signal provides the sensitivity necessary to measure haemoglobin, pulse-rate, oxymetry (even under severe low perfusion levels), and other analyte concentrations.

Vein finder: The principle involves the use of near infrared light to highlight deoxygenated haemoglobin in a patient's veins and capture the images with two stereoscopic cameras. The cameras then project the vein images onto the display visual display unit. Visualization of subcutaneous structures will increase the speed and accuracy with which medical treatments requiring insertion of instruments into these structures can be performed. The central database can then store the images or videos and transfer them wirelessly to a patient's electronic health record. Further, a simpler alternative works by using near-infrared wavelength LEDs to illuminate the flesh at point of contact. The veins will appear as dark bands because they are more absorbent of this spectrum of light than the surrounding tissue.

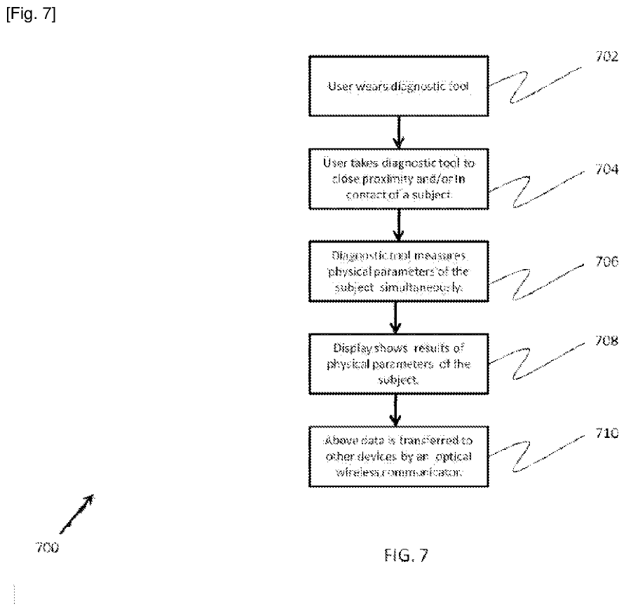

FIG. 7 shows the flowchart 700 illustrating a method of measuring physical parameters of a subject by a user. Said method involves user wearing the diagnostic device 702, where the user can be any professional (doctor or any medical professional) who needs to examine physical parameters of a subject. The method of measuring physical parameters further involves the user taking the diagnostic device in close proximity of a subject 704. For the purposes of this document, the term ‘close proximity’ shall include taking the diagnostic device close to the subject wherein the diagnostic device may or may not make direct contact with the subject depending upon the nature of physical parameter being measured and the strength of the sensors incorporated within the diagnostic device. Various physical parameters like heart rate, oxygen saturation, body temperature, blood pressure etc. of the subject may be measured simultaneously by various sensors embedded within the diagnostic device 706. These measured parameters are exhibited on a display disposed on the diagnostic device 708. In one embodiment, the health data comprising various physical parameters can be transferred to other devices 710 using optical wireless communication method and processed further.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practised with modification within the spirit and scope of the embodiments as described herein.

1. A wearable diagnostic device comprising:
an apparel;
a modular sensor matrix detachably disposed on said apparel wherein said modular sensor matrix enables a user to measure at-least one physical parameter of a subject;
an input channel disposed on said modular sensor matrix wherein said channel is configured to provide desired input variables to said modular sensor matrix; and
a visual display unit detachably disposed on said apparel wherein said visual display unit is configured to receive data from said modular sensor matrix and exhibit said physical parameter of said subject.

2. The system of claim 1 wherein said apparel is a glove, wrist band, sleeve and the like.

3. The system of claim 1 wherein said physical parameters comprises of blood sugar level, oxygen saturation, pulse rate, foetal heart rate, haemoglobin, wheezing, cardiac murmur, body temperature, anaemia and the like.

4. The system of claim 1 wherein said modular sensor matrix comprises of blood sugar level measuring sensor, oxygen saturation measuring sensor, pulse rate measuring sensor, foetal heart rate measuring sensor, haemoglobin measuring sensor, wheezing measuring sensor, cardiac murmur measuring sensor, body temperature measuring sensor, anaemia measuring sensor and the like.

5. The system of claim 1 wherein said input variables comprises of frequency, wavelength, speed and the like.

6. The system of claim 1 wherein said modular sensor matrix is configured to simultaneously measure more than one physical parameters of said subject.

7. The system of claim 1 wherein said visual display unit is configured to simultaneously display more than one physical parameter measurement of said subject.

8. The system of claim 1 wherein said system further comprises an integrated camera wherein said camera is configured to function as an X-ray scanner.

9. The system of claim 1 wherein said system further comprises an optical wireless communication module for data transmission.

10. The system of claim 1 wherein said system further comprises at-least one support configured to attach said visual display unit and said sensor matrix to said apparel.

11. A method for measuring physical parameters of a subject by a user using a wearable diagnostic device, said method comprising:
taking said wearable diagnostic device in substantially close proximity of said subject wherein said wearable diagnostic device comprising of, an apparel;
a modular sensor matrix detachably disposed on said apparel;
an input channel disposed on said modular sensor matrix; and
a visual display unit detachably disposed on said apparel;
measuring at-least one physical parameter of said subject by said user using said modular sensor matrix disposed on said diagnostic device; inputting at-least one variable using said input channel disposed on said modular sensor matrix; and
exhibiting said physical parameter measurements of said subject on said visual display unit disposed within said diagnostic tool.

12. The method of claim 11 wherein said apparel is a glove, wrist band, sleeve and the like.

13. The method of claim 11 wherein said physical parameters comprises of blood sugar level, oxygen saturation, pulse rate, foetal heart rate, haemoglobin, wheezing, cardiac murmur, body temperature, anaemia and the like.

14. The method of claim 11 wherein said modular sensor matrix comprises of blood sugar level measuring sensor, oxygen saturation measuring sensor, pulse rate measuring sensor, foetal heart rate measuring sensor, haemoglobin measuring sensor, wheezing measuring sensor, cardiac murmur measuring sensor, body temperature measuring sensor, anaemia measuring sensor and the like.

15. The method of claim 11 wherein said input variables comprises of frequency, wavelength, speed and the like.
